(19) United States
(12) Patent Application Publication
(10) Pub. No.: US 2003/0026779 A1
(43) Pub. Date: Feb. 6, 2003

(54) Treatment of tumors and viral infections with a hybrid conjugate of interferon and an immunoglobulin Fc

(76) Inventors: Liming Yu, Piscataway, NJ (US); Tse Wen Chang, Hsinchu (TW)

Correspondence Address: TANOX, INC., 10301 STELLA LINK, HOUSTON, TX, 77025, US

(21) Appl. No.: 10/005,438

(22) Filed: Dec. 3, 2001

Related U.S. Application Data
(63) Continuation of application No. 09/418,734, filed on Oct. 15, 1999, now abandoned.

(51) Int. Cl. A61K 39/395; C07K 16/46

(52) U.S. Cl. 424/85.4; 424/155.1; 530/351

(57) ABSTRACT

The present invention relates to interferon-immunoglobulin Fc fusion proteins (referred to as “IFN-Fc hybrids”) and their use in treating tumors. The IFN-Fc hybrids preferably (but not necessarily) include linkers between the IFN and the Fc portion, and the IFN portion can be an IFN variant. These linkers are preferably composed of a T cell inert sequence, or any non-immunogenic sequence, including Gly-Ser repeat units. The preferred Fe fragment is a human immunoglobulin Fc fragment, preferably the γ4 chain.

### RELATED APPLICATIONS

This application claims priority to U.S. application Ser. No. 08/994,719, filed Dec. 19, 1997 (now U.S. Pat. No. 5,908,626), which is a continuation-in-part of U.S. application Ser. No. 08/719,331, filed Sep. 25, 1996 (now U.S. Pat. No. 5,723,125) which is a continuation-in-part of U.S. application Ser. No. 08/579,211, filed Dec. 28, 1995 (now abandoned).

### FIELD OF THE INVENTION

This invention relates to novel interferon hybrid proteins, in which an interferon is conjugated with an immunoglobulin Fc, for treating tumors and viral infections.

### BACKGROUND OF THE INVENTION

Interferons, including interferon-α (“IFNα”) and interferon-β (“IFNβ”), were among the first of the cytokines to be produced by recombinant DNA technology. IFNα has been shown to have therapeutic value in conditions such as hairy cell leukemia, and inflammatory and viral diseases, including hepatitis B. IFNβ has been approved for use in treatment of multiple sclerosis.

Most cytokines, including IFNα, have relatively short circulation half-lives since they are produced in vivo to act locally and transiently. To use IFNα as an effective systemic therapeutic, one needs relatively large doses and frequent administrations. Such frequent parenteral administrations are inconvenient and painful. Further, toxic side effects are associated with IFNα administration are so severe that some cancer patients cannot tolerate the treatment. These side effects are probably associated with administration of a high dosage.

To overcome these disadvantages, one can modify the molecule to increase its circulation half-life or change the drug's formulation to extend its release time. The dosage and administration frequency can then be reduced while increasing the efficacy. Efforts have been made to create a recombinant IFNα-gelatin conjugate with an extended retention time (Tabata, Y. et al., Cancer Res. 51:5532-8, 1991). A lipid-based encapsulated IFNα formulation has also been tested in animals and achieved an extended release of the protein in the peritoneum (Bonetti, A. and Kim, S. Cancer Chemother Pharmacol. 33:258-261, 1993).

Immunoglobulins of IgG and IgM class are among the most abundant proteins in the human blood. They circulate with half-lives ranging from several days to 21 days. IgG has been found to increase the half-lives of several ligand binding proteins (receptors) when used to form recombinant hybrids, including the soluble CD4 molecule, LHR, and the IFN-γ receptor (Mordenti J. et al., Nature, 337:525-31, 1989; Capon, D. J. and Lasky, L. A., U.S. Pat. No. 5,116,964; Kurschner, C et al., J. Immunol. 149:4096-4100, 1992). The invention relates to using IFNα-Fc hybrids, which may or may not include peptide linkers between the IFNα and the Fc portion, for treatment of tumors.

### SUMMARY OF THE INVENTION

The present invention relates to IFN-Fc hybrids and their use in treating tumors and viral infections. The IFN hybrids can be IFNα-Fc or IFNβ-Fc hybrids. The IFNα-Fc or IFNβ-Fc in the hybrid include variants, including the IFNβ variant in Betaseron™. The hybrids preferably (but not necessarily) include peptide linkers between the IFN and the Fc portion. These linkers are preferably composed of a T cell inert sequence, or any non-immunogenic sequence. The preferred Fc fragment is a human immunoglobulin Fc fragment, preferably the γ4 chain. The γ4 chain is preferred over the γ1 chain because the former demonstrates little or no antibody-dependant cell-mediated cytotoxicity (ADCC), complement activating ability and is stable in human circulation.

In one embodiment, the C-terminal end of the IFN is linked to the N-terminal end of the Fc fragment. An additional IFN (or other cytokine) can attach to the N-terminal end of any other unbound Fc chains in the Fc fragment, resulting in a homodimer, if the Fc selected is the γ4 chain. If the Fc fragment selected is another chain, such as the μ chain, then, because the Fc fragments form pentamers with ten possible binding sites, this results in a molecule with interferon, or another cytokine, linked at each of ten binding sites.

The two moieties of the hybrid are preferably linked through a T cell immunologically inert peptide including, for example, peptides with Gly Ser repeat units. Because these peptides are immunologically inactive, their insertion at the fusion point eliminates any neoantigenicity which might have been created by the direct joining of the IFNα-Fc moieties.

The IFNα-Fc hybrids of the invention are predicted to have a much longer half-life in vivo than the native IFNα, and this is supported by experimental data. Cytokines are generally small proteins with relatively short half-lives which dissipate rapidly among various tissues, including at undesired sites. It is believed that small quantities of some cytokines can cross the blood-brain barrier and enter the central nervous system, thereby causing severe neurological toxicity. The IFN-Fc hybrids of the present invention would be especially suitable for treating tumors, including lymphomas and leukemias, because these products will have a long retention time in the vasculature and will not penetrate undesired sites.

The IFN-Fc hybrids can be administered in a pharmaceutical formulation including suitable excipients and additives. The dosage for human use can be readily determined by extrapolation from animal data, with compensation for differences in size, and routine experimentation in clinical trials.

### BRIEF DESCRIPTION OF THE SEQUENCE LISTING

SEQ ID NO:1 is the nucleotide sequence of an IFN-α-Fc hybrid, with no linker.

SEQ ID NO:2 is the amino acid sequence of an IFN-α-Fc hybrid shown in SEQ ID NO:1.

SEQ ID NOS:3-9 are the amino acid sequences of the various length peptide linkers used to conjugate the N-terminal end(s) of a heavy chain γ4 Fc fragment to the C-terminal end of an IFN-β molecule.

SEQ ID NO:10 is the amino acid sequence of a linker used to conjugate the N-tenninal end of a heavy chain γ1 Fc fragment to the C-terminal end of an IFN-α, as used in an assay as described below.

SEQ ID NO:11 is the amino acid sequence of a linker used to conjugate the N-terminal end of a heavy chain γ4 Fc fragment to the C-terminal end of an IFN-α, which molecule was then used in an in vitro cytopathic effect assay as described below.

### BRIEF DESCRIPTION OF THE DRAWINGS

FIG. 1 shows a virus cytopathic effect inhibition assay for various linkers in an IFN-β-Fc hybrid.

FIG. 2 shows a virus cytopathic effect inhibition assay for two different linkers in an IFN -α-Fc hybrid.

### DETAILED DESCRIPTION OF MAKING AND USING THE INVENTION

The preferred hybrid molecules of the invention have C-terminal ends of two interferon moieties separately attached (and more preferably, attached through a linker) to each of the two N-terminal ends of a heavy chain γ4 Fc fragment, resulting in a homodimer structure. Any of a number of immunologically inert linker peptides, including those with a Gly Ser repeat unit, can link the two moieties. Alternatively, no linker can be used.

The complete nucleotide sequence of an IFNα-Fc hybrid with no linker appears in SEQ ID NO: 1 and the amino acid sequence is shown in SEQ ID NO:2. The linker, if present, is located between amino acid residue numbers 188 (Glu) and 189 (Glu). The sequences of a number of suitable linkers which were all shown to have about the same cytopathic effect in vitro, are shown in SEQ ID NOS: 3 to 8. Any of a number of other linkers can also be used. Alternatively, no linker can be used.

One significant advantage of the hybrid of the invention over the native cytokine is that the hybrids of the invention have been shown to ablate tumors in an animal model, described below. IFN-α itself is approved for use in treating certain tumors and hepatitis B. The hybrids of the invention may also work more effectively in treating infectious diseases and a broad range of tumors than IFNα itself.

The cDNA of the IFNα can be obtained by reverse transcription and PCR, using RNA extracted from cells which express IFNα, and following the extraction with reverse transcription and expression in a standard expression system. There are several ways to express the recombinant protein in vitro, including in E. coli, baculovirus, yeast, mammalian cells or other expression systems. The prokaryotic system, E. coli, is not able to do post-translational modification, such as glycosylation. This could be a problem in these systems, and mammalian expression could be preferred for this reason, and because it offers other advantages in terms of simplifying purification.

There are several assay methods available for the measuring of the IFNα bioactivity, including an antiviral assay. The hybrids of the invention have a longer half-life in vivo than native IFNα based on in vitro experimental results, described below. Even though the specific activity is lower, the hybrids of the invention are preferred to the native IFNα for clinical use. This is because, as a result of the longer half-life, the Cxt (the area under the concentration vs. time curve) is much greater, based on in vitro results than for the native IFNα. This means that at the equivalent molar dosage of the native IFNα and the hybrid, the latter would provide a several hundred fold increased exposure to IFNα, resulting in vastly increased efficacy at the same dosage, and less frequent administration. The invention will now be described with reference to examples and experimental results.

### EXAMPLE I

### IFNα-Fc Hybrid Demonstrates a Large Increase in Half-Life over the Native IFNα.

The disclosures of U.S. Pat. No. 5,723,125 (incorporated by reference) describes making an IFNα-Fc(γ1) hybrid with a linker having the sequence: Gly Gly Ser Gly Gly Ser (SEQ ID NO:10). The specific activity of this hybrid was 7.7×10⁸ units/μmole in an in vitro assay in Daudi cells, compared with 15.4×10⁸ units/μmole for the unmodified interferon-cc in the same assay. In a later cytopathic effect inhibition assay, the hybrid showed an antiviral specific activity of 2.2×10⁸ IU/μmole, which is lower than the 3.8×10⁹ IU/μmole of the unmodified interferon-α. In attempting to increase the specific activity of the hybrid, the linker was extended, to increase the flexibility and decrease steric hindrance. A linker having the sequence: Gly Gly Ser Gly Gly Ser Gly Gly Gly Gly Ser Gly Gly Gly Gly Ser (SEQ ID NO:11) was used. Another difference in the new hybrid was that the Fc portion was γ4 Fc, rather than γ1Fc.

The results of a virus cytopathic effect inhibition assay, in vitro, showed that the new hybrid had an antiviral specific activity of 1.1-2.2×10⁹ IU/μmole, a 5-10 fold increase over the old one. Nevertheless, it is still 2-3 fold less than that of the unmodified interferon-α, which had a specific activity of 3.8×10⁹ IU/μmole in this same assay. However, in an in vivo pharmacokinetic study in primates, the serum half-life of the claimed new hybrid was about 40 fold longer than the unmodified interferon. Also, the clearance half-life after subcutaneous (s.c.) administration of the hybrid was almost 120 fold longer. The hybrid, when administered s.c., was also well absorbed. The large increase in the AUC (area under curve) for the new hybrid means that it likely would be more efficacious than native interferon-α, notwithstanding its lower specific activity.

Experiments described below were then conducted to determine the effect of using linkers of different lengths on cytopathic activity.

### EXAMPLE II

### Study of the Effect of Various Linkers on IFN-Fc Cytopathic Activity.

1. Comparison of IFN-α(16)Fc and IFN-α-Ala-Fc

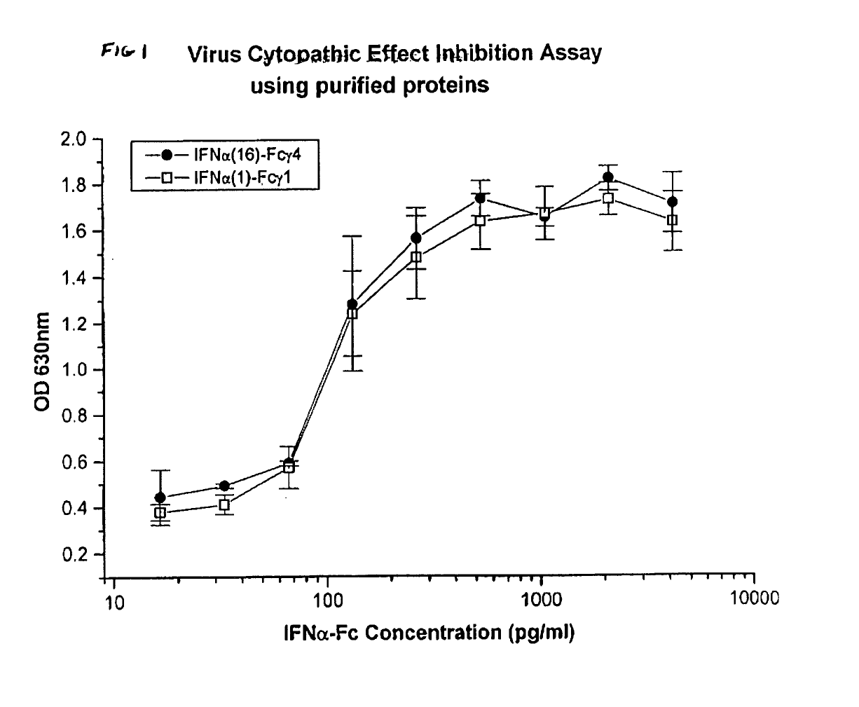

The effect of linker peptides was tested by comparing IFN-α(16)Fc and IFNα-Ala-Fc. IFN-α(16)Fc contains IFNα linked to the hinge region of the human IgG4 Fc through the 16-amino acid linker shown in SEQ ID NO:11, i.e. GlyGlySerGlyGlySerGlyGlyGlyGlySerGlyGlyGlyGlySer. The IFN-α-Ala-Fc construct contains IFN-α linked to the hinge region of the human IgG1 Fc with one amino acid (Ala) between the two domains. DNA fragments encoding IFN-α(16)Fc and IFN-α-Ala-Fc were inserted, respectively, at the polycloning site of the pcDNA3 expression plasmid. Purified plasmid DNA was then used to transfect NS0 cells by electroporation. Stably-transformed cell lines were selected in the presence of G418. Cell lines expressing these linker variants were then grown in spinner culture flasks. Spent culture supernatant was collected and purified proteins were prepared using the protein A affinity column. Purified proteins were used in the same virus cytopathic effect inhibition assays as described in Example I. Both IFN-α-Ala-Fc and IFN-α(16)Fc were shown to have equivalent activities (FIG. 1).

2. Constructs for IFNβ-Fc linker variants

A number of different constructs of interferon-β linked to an Fc (“IFNβ-Fc”) were made, to determine the effect of linker length on the activity of the IFNβ-Fc hybrid. The amino acid sequences of these constructs are listed in the following Table 1.

Table 1
Translated amino acid sequences of various IFNβ-Fc.

| | |
|---|---|
| Linker Variants | Linker Sequence between IFNβ and the hinge of IgG4(Fc) |

| | | |
|---|---|---|
| IFNβ-(2)Fc | GlySer | (SEQ ID NO:3) |
| IFNβ-(8)Fc | GlyGlyGlySerGlyGlyGlySer | (SEQ ID NO:4) |
| IFNβ-(12)Fc | GlySerGlyGlyGlyGlySerGlyGlyGlyGlySer | (SEQ ID NO:5) |
| IFNβ-(18)Fc | GlyGlyGlySerGlyGlyGlySerGlyGlyGlyGlySerGlyGlyGlyGlySer | (SEQ ID NO:6) |
| IFNβ-(23)Fc | GlyGlyGlySerGlyGlyGlySerGlyGlyGlyGlySerGlyGlyGlyGlySerGly | (SEQ ID NO:7) |
| | GlyGlyGlySer | |
| IFNβ-(30)Fc | GlyGlyGlySerGlyGlyGlySerGlyGlyGlyGlyGlySerGlyGlyGlyGlyGly | (SEQ ID NO:8) |
| | SerGlyGlyGlyGlySerGlyGlyGlyGlySer | |
| IFNβ-(40)Fc | GlyGlyGlySerGlyGlyGlySerGlyGlyGlyGlyGlySerGlyGlyGlyGlyGly | (SEQ ID NO:9) |
| | SerGlyGlyGlyGlySerGlyGlyGlyGlySerGlyGlyGlyGlySerGlyGlyGly | |
| | GlySer | |

3. Expression of IFNβ-Fc linker variants

DNA sequences containing different IFNβ-Fc linker variants were inserted, respectively, at the polycloning site of the pcDNA3 expression plasmid. Purified DNA was then used to transfect NS0 cells by electroporation. Stably-transformed cell lines were initially selected in the presence of G418. Cell lines expressing these linker variants were then grown in the absence of G418. Spent culture supernatant was collected and filtered through a 0.22 μm membrane. The concentration of IFNβ-Fc was estimated by PCFIA using purified IFNβ-Fc protein as the standard. Concentrations of culture supernatant were estimated to be 5.4, 22.5, 15.9, 5.7, 10.2, 5.5, and 4.5 μg/ml for the IFNβ-Fc variants containing linker peptides of 2, 8, 12, 18, 23, 30 and 40 amino acids, respectively. These supernatants were used in the following in vitro cytopathic effect experiments.

4. In vitro cytopathic effect assays using the IFNβ-Fc variants.

In 96-well plates, human lung carcinoma A549 cells were plated at 100 μl/well containing 5×10⁴ cells using DMEM containing 5% FBS. Plates were incubated at 37° C. for 24 hrs in the 5% CO₂ incubator. Culture supernatants containing the IFNβ-Fc linker variants were diluted. These solutions were then used to make 2-fold serial dilutions in a 96-well plate using DMEM containing 5% FBS. One hundred μl of the diluted samples were added to each well and the plates were incubated at 37° C. for an additional 24 hours in the incubator. Culture supernatant was removed and encephalomyocarditis (EMC) virus was added at 100 μl/well (the virus is diluted 1:200 in D15 containing 5% FBS from virus stock). The plates were then incubated at 37° C. for 48hrs in the 5% CO₂ incubator. Culture supernatant was removed and the wells were washed 2 times with PBS. Cells were then fixed with paraformaldehyde; and stained with the giemsa dye, then left for 5 minutes at room temperature. Thereafter, the plates were rinsed gently with tap water several times. Methanol was added to each well and the wells were read at 630 nm using the Dynatech MR5000 ELISA reader.

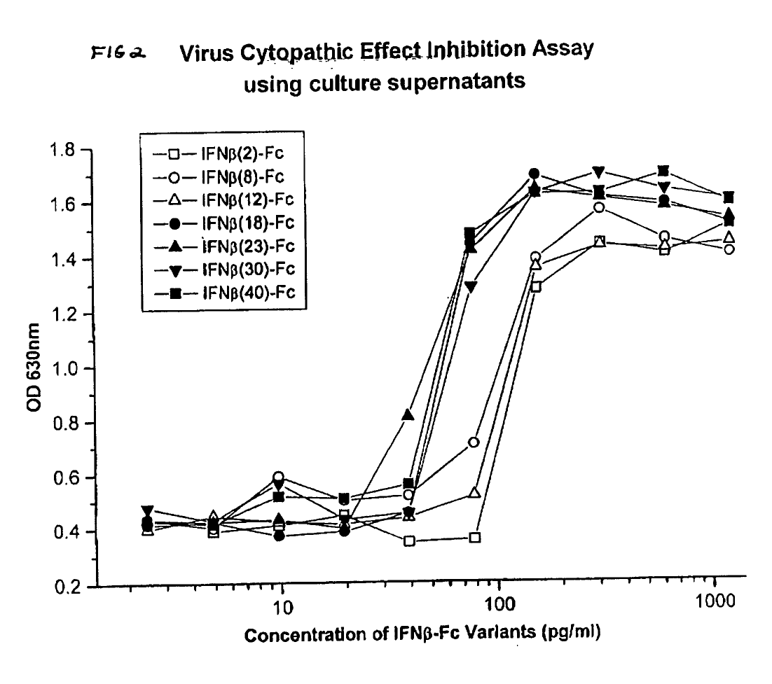

The results of several experiments with IFNβ-Fc hybrids, as shown in FIG. 2, and the results for the two different IFNα-Fc hybrids shown under heading 1 of this Example II, show that the cytopathic effect did not change significantly no matter which linker was used. Further in vivo experiments on one of the IFNα-(16)Fc hybrids were conducted as described below.

### EXAMPLE III

### Animal Tumor Model.

1. Tumor Initiation in Mice.

Female CB17/scid mice (Charles River Laboratories; seven and half weeks old) were inoculated subcutaneously (s.c.) with Daudi Burkitt lymphoma cells at the lower right flank at a total volume of 100 μl. There were four different cell densities tested in five animals in each group (Table 2). The injection site was monitored one day after inoculation and then daily three weeks after inoculation.

Palpable tumors were measured by caliper. Tumor volume was determined and calculated using the formula, V=4 xyz/3, where 2x, 2y and 2z are the three perpendicular diameters of the tumor and the average of two measurements.

For inoculation, cells were grown in vitro in D15 media with 10% fetal calf serum in 100 ml spinners to a density of 0.6×10⁶/ml with 94% viability. Cells were harvested by centrifugation at 300 g for 10 minutes, washed twice in cold PBS, and resuspended to the desired density in PBS. Cell counting and Tryptan Blue staining confirmed the cell density and viability.

TABLE 2
Cell Density and Route of Inoculation

| Cell Density | No. of Animals | Route of Administration |
|---|---|---|
| PBS | 5 | s.c. |
| 0.5 × 10⁶/100 ul PBS | 5 | s.c. |
| 2.5 × 10⁶/100 ul PBS | 5 | s.c. |
| 1.25 × 10⁷/100 ul PBS | 5 | s.c. |

Human tumor xenografts became detectable in the 125×10⁷ group at the site of injection four weeks after inoculation. One week later, the tumor take rate reached 80% and was maintained at this level throughout the entire pilot study period. It took about three weeks (2.5-3.5 wks) for a palpable tumor to grow up to 10-15% of the animal's body weight. In the 2.5×10⁶ and 0.5×10⁶ groups, the take rate reached 60% by the end of the nine and half weeks. The tumors did not kill the mice and there was no sign of metastases.

Thus, it is concluded that a subcutaneous inoculation of 1.25×10⁷ Daudi Burkitt lymphoma cells will yield about 80% tumor takes in about four weeks.

2. In vivo antiproliferation Study

1. Experiment with daily dosing

Thirty-two mice inoculated with 12.5×10⁶ Daudi Burkitt lymphoma cells were randomly assigned to one of four treatment groups as shown in Table 3. Roferon A (IFN-α-2a, Hoffmann La Roche, Nutley, N.J.) and IFN-α(16)-2a-Fc (having the linker shown in SEQ ID NO:11) treatment began the day after tumor inoculation. All the animals were dosed daily subcutaneously over the scruff and the treatment continued for eight consecutive weeks. During the treatment period, animals were monitored every 3-4 days for tumor development, and tumor size was measured as above. After the treatment period, weekly observations were continued for additional six months for animals that were tumor free by the time when treatment stopped.

Blood was collected retro-orbitally 24 hours post the last dosing day, one, two and four weeks after termination of the treatment for IFN-α-2a-Fc and one, two and three weeks after termination of Roferon A treatment. Serum Interferon level was determined by ELISA.

TABLE 3
Dose, route and schedule

| | | Route of | |
|---|---|---|---|
| Group | Dose | Administration | Schedule |
| Control | Diluent | s.c. | daily |
| Roferon A | 1 × 10⁶ IU/100 μl | s.c. | daily |
| IFN-α-Fc | 1 × 10⁶ IU/100 μl | s.c. | daily |
| IFN-α-Fc | 1 × 10⁵ IU/100 μl | s.c. | daily |

2. Effect of IFN-α on tumor take rate and tumor progression

Tumor development in different treatment groups is shown in Table 4. In control animals, the first tumor was detected 24 days after inoculation and within 6 days thereafter 7/8 (87.5%) of the animals had developed tumors. The average time of tumor detection was 25.1±2.3 days (The mouse that developed a tumor at day 75 was not included.). In Roferon A treated animals, the first tumor became detectable 32 days after the inoculation. After another two weeks, 87.5% had developed tumors. The average tumor detection time was 39.6±4.7 days (t>t _{0.05 (12)}, P<0.05). Roferon A delayed tumor development for about two weeks. IFN-α-2a-Fc treatment at both doses completely prevented the Daudi lymphoma from developing throughout the entire dosing period. At the lower dose, two mice developed detectable tumors at 2 and 19 days after cessation of the treatment. While all mice in 1×10⁶ IU/day group and the remaining six mice in 1×10⁵ IU/daily still remained tumor free six months post treatment. (Table 4). This experiment was repeated once with similar results, as shown in Table 4.

TABLE 4
Tumor Development in CB17/scid Mice (Exp.1.)

| | | | Tumor | |
|---|---|---|---|---|
| Mouse | Date of | Date of | Development/ | Mean ± |
| I.D. | Inoculation | Tumor Detection | Time (days) | S.D. |
| C* 116 | May 27, 1998 | Jun. 20, 1998 | 24 | |
| 117 | May 27, 1998 | Jun. 20, 1998 | 24 | |
| 125 | May 25, 1998 | Jun. 20, 1998 | 24 | |
| 134 | May 27, 1998 | Jun. 20, 1998 | 24 | |
| 114 | May 27, 1998 | Jun. 20, 1998 | 24 | |
| 101 | May 27, 1998 | Jun. 22, 1998 | 26 | |
| 119 | May 27, 1998 | Jun. 26, 1998 | 30 | 25.1 ± |
| | | | | 2.3 |
| R* 133 | May 27, 1998 | Jun. 28, 1998 | 32 | |
| 104 | May 27, 1998 | Jul. 1, 1998 | 35 | |
| 103 | May 27, 1998 | Jul. 6, 1998 | 40 | |
| 115 | May 27, 1998 | Jul. 6, 1998 | 40 | |
| 110 | May 27, 1998 | Jul. 7, 1998 | 41 | |
| 113 | May 27, 1998 | Jul. 9, 1998 | 43 | |
| 128 | May 27, 1998 | Jul. 12, 1998 | 48 | 39.6 ± |
| | | | | 4.7 |

*C indicates a control
*R indicates that Roferon A was administered at 1 × 10^{6 IU/day}

3. Effect of IFN-α on tumor growth rate

Once the tumor grew to about 1% of the mouse's body weight, tumor growth rate in control and Roferon A treated animals were very close. In control animals, average tumor volume increased 10 times in two weeks, while Roferon A treated mice showed a 9-fold increase.

TABLE 5
Tumor Take Rate in Different Treatments

| | | Tumor Take Rate (%) |
|---|---|---|
| Group | Treatment | (N = 8) |
| Control | Diluent | 100 (8/8) |
| Roferon A | 1 × 10⁶ IU/100 ul | 87.5 (7/8) |
| IFN α-2a-Fc | 1 × 10⁶ IU/100 ul | 0 |
| IFN α-2a-Fc | 1 × 10⁵ IU/100 ul | 25.0 (2/8) |

4. Quantitation of serum IFN-α level

Serum concentration of IFN-α and IFN-α-2a-Fc was determined by ELISA procedures. In Roferon A treated mice, IFN-α -2a was undetectable 24 hours after the last dose. In IFN-α-2a-Fc treated mice, serum IFN-α-2a-Fc concentration was 3.5 ug/ml for the 1×10⁶ IU/day group and 0.7 ug/ml for the 1×10⁵ IU/day group 22 days after termination of the treatment (Table 6). There was a decrease in serum concentration between 1 and 22 days after the end of the treatment. The data indicate that IFN-α-2a-Fc has a half-life of about one week in mice after being administered subcutaneously 1×10⁶ IU/day or 1×10⁵ IU/day for 8 weeks.

TABLE 6
Serum IFN-α-2a Level (μg/ml)

| | Days Post Treatment Termination | | |
|---|---|---|---|
| Treatment | 1 | 8 | 22 |
| IFN-α-2a-Fc 1 × 10⁶ | 25.370 ± 6.885 | 12.080 ± 3.477 | 3.477 ± 0.525 |
| IU | | | |
| IFN-α-2a-Fc 1 × 10⁵ | 2.766 ± 1.138 | 1.549 ± 0.536 | 0.691 ± 0.141 |
| IU | | | |
| Roferon A | Undetectable | Undetectable | Undetectable |

5. Experiment with an increased-dosing-interval

In this experiment, Roferon A 1×10⁶ IU was given every 3 days and 1×10⁶ IU IFN-α-2a-Fc was dosed every three days and weekly. The results are shown in Table 7. Roferon A 1×10⁶ IU for 3 days failed to show any protection against tumor formation as compared to the control animals in tumor volume and average time for tumor development, while 1×10⁶ IU IFN-α-Fc administered every three days and weekly effectively inhibited the tumor formation during the eight week treatment period. This inhibition extended to seven weeks after the treatment period.

TABLE 7
Tumor Development in animals with an increased dosing intervals

| | Tumor Take | Average Time for Tumor |
|---|---|---|
| | Rate (%) | Development |
| Treatment | (N = 8) | (days) |
| Control | 100 (8/8) | 21.1 ± 1.1 |
| Roferon A 10⁶ IU/3 days | 100 (8/8) | 22.0 ± 1.9 |
| IFN-FC 10⁶ IU/3 days | N/A | N/A |
| IFN-FC 10⁶ IU/weekly | N/A | N/A |

7. Preliminary study with established Daudi Burkitt lymphomas

Two mice with well established 5-week-old Daudi Burkitt lymphomas were treated with IFN-α-Fc at 10⁶ IU/daily. After ten days, complete regression was observed in both of the animals (Table 8). Two other mice with established 6.5-week-old Daudi lymphomas were treated with 10⁶ IU Roferon A every three days for eight weeks. In the latter mice, tumor volume decreased rapidly, declining from 2.7cm³ and 4.6 cm³ to 0.3 cm³, a reduction of 89% to 94% in the first two weeks. Complete regression was not achieved.

TABLE 8
Tumor Regression in Control Mice

| Mouse I.D. | Date | Tumor Volume (cm³) |
|---|---|---|
| 416 | Nov. 20, 1998 | 0.195 (7 mm × 7.6 mm) |
| | Nov. 24, 1998 | 0.161 (6.4 mm × 7.6 mm) |
| | Nov. 25, 1998 | palpable |
| | Nov. 26, 1998 | palpable |
| | Nov. 27, 1998 | complete regression |
| 453 | Nov. 20, 1998 | 0.858 (10 mm × 16 mm) |
| | Nov. 24, 1998 | 0.393 (6.4 mm × 7.6 mm, 7.6 mm × 7.6 |
| | | mm) |
| | Nov. 25, 1998 | palpable |
| | Nov. 26, 1998 | palpable |
| | Nov. 27, 1998 | palpable |
| | Nov. 28, 1998 | barely palpable |
| | Nov. 29, 1998 | complete regression |

Summary and Conclusions

1. IFN-α-2a-Fc hybrids with linkers of one amino acid or 16 amino acids demonstrated equivalent activity in a virus cytopathic assay.

2. IFN-β-Fc hybrids with a wide variety of linker lengths demonstrated similar effects in a viral cytopathic assay.

3. Roferon A 1×10⁶ IU/day treatment delayed the Daudi B cell lymphoma development by two weeks (t>t _{0.05 (12)}, P<0.05). IFN-α-2a-Fc 1×10⁶ IU/day completely inhibited the tumor formation throughout the entire dosing period and this inhibition has been extended to six months after termination of the treatment. Partial to full inhibition was also shown in the 1×10⁵ IU /day IFN-α-2a-Fc treated mice.

4. Roferon A 1×10⁶ IU/3 days treatment failed to show any protection against the tumor development whereas Daudi Burkitt lymphoma has been completely inhibited by either IFN-α-Fc at 1×10⁶ IU/3 days or the IFN-α-2a-Fc 1×10⁶ IU/weekly, and inhibition continued for at least seven weeks after cessation of the treatment.

5. Preliminary data demonstrated that established, 5-week-old Daudi Burkitt lymphomas are completely regressed when treated with IFN-α-2a-Fc 10⁶ IU/daily for ten days. A 90% reduction of tumor volume in 2 weeks is also achieved in Daudi Burkitt lymphomas which were treated with 10⁶ IU Roferon Alevery 3 days for seven weeks before the IFN-α-2a-Fc treatment started.

It should be understood that the terms and expressions used herein are exemplary only and not limiting, and that the scope of the invention is defined only in the claims which follow, and includes all equivalents of the subject matter of those claims.

What is claimed is:

1. A method of treating tumors or viral infections comprising administering a hybrid molecule having an interferon molecule, or a variant thereof, joined at one end to a first end of an immunoglobulin Fc fragment, without any linker between the interferon and the immunoglobulin Fc fragment.

2. A method of treating tumors or viral infections comprising administering a hybrid molecule having an interferon molecule, or a variant thereof, joined at one end to a first end of an immunoglobulin Fc fragment with a first linker between the interferon and the immunoglobulin Fc fragment.

3. The hybrid molecule of claim 1 wherein the interferon molecule is joined at its C-terminal end to the N-terminal end of an immunoglobulin Fc fragment.

4. The hybrid molecule of claim 2 wherein the interferon molecule is joined at its C-termiinal end through the first linker to the N-termninal end of an immunoglobulin Fc fragment.

5. The hybrid molecule of claims 1 wherein another interferon molecule is joined at its end to the end of the other chain of the imimunoglobulin Fc fragment, thereby forming a homodimer.

6. The hybrid molecule of claims 2 wherein another interferon molecule is joined at its end through a second linker to the end of the other chain of the immunoglobulin Fc fragment, thereby formning a homodimer.

7. The hybrid molecule of claims 1 or 2 wherein the Fc fragment is a γ4 chain Fc fragment.

8. The hybrid molecule of claim 2 wherein the linker comprises Gly-Ser repeat units.

10. The hybrid molecule of claim 9 wherein the linker is between two and 40 amino acids in length.
